(19) United States
(12) Patent Application Publication
(10) Pub. No.: US 2008/0181953 A1
(43) Pub. Date: Jul. 31, 2008

(54) COMPOSITION CONTAINING A TENSIONING POLYMER AND HYBRID PARTICLES, METHODS OF USE

(75) Inventor: Guillaume CASSIN, Villebon Sur Yvette (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/970,560

(22) Filed: Jan. 8, 2008

Related U.S. Application Data
(60) Provisional application No. 60/885,033, filed on Jan. 16, 2007.

(30) Foreign Application Priority Data
Jan. 9, 2007 (FR) 07 52577

(51) Int. Cl. A61K 31/74; A61K 9/14; A61P 17/00

(52) U.S. Cl. 424/486; 424/78.08; 424/78.17

(57) ABSTRACT

Composition containing, in a physiologically acceptable medium, a tensioning polymer and organic-mineral hybrid particles. Process, particularly for facial skin, in particular wrinkled skin, involving the application of the invention composition to the skin.

### REFERENCE TO PRIOR APPLICATIONS

This application claims priority to U.S. provisional application No. 60/885,033 filed Jan. 16, 2007, and to French patent application 0752577 filed Jan. 9, 2007, both incorporated herein by reference.

### FIELD OF THE INVENTION

The present invention relates to a composition, especially an anti-wrinkle composition, comprising, in a physiologically acceptable medium, a tensioning polymer and organic-mineral hybrid particles. The invention also relates to a process for caring for wrinkled skin, for the purpose of attenuating wrinkles, comprising the application to the skin of a composition as defined above.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BRIEF DESCRIPTION OF THE DRAWING

FIG. 1 shows an example of a curve of force as a function of displacement.

### BACKGROUND OF THE INVENTION

During the ageing process, impairment of the structure and functions of the skin appears. The main clinical signs observed are the appearance of wrinkles and fine lines associated with slackening of the skin. A person skilled in the art knows that such slackening may be corrected immediately by applying a tensioning agent to the skin.

The use of numerous tensioning polymers for treating wrinkles is known to those skilled in the art at the present time. Acrylic-grafted silicone polymers or interpenetrating polymer networks for smoothing out wrinkles via a tensioning effect, described especially in documents EP 1 038 519 and FR 2 843 025, are recalled in particular.

Unfortunately, although compositions containing such tensioning agents have a very satisfactory immediate tensioning effect, they have the major drawback of having a limited effect over time.

### SUMMARY OF THE INVENTION

Consequently, there is a need for compositions that have both excellent efficacy and a long-lasting tensioning effect. Now, the inventor has discovered that the inclusion of organic-mineral hybrid particles into compositions comprising a tensioning polymer makes it possible to improve the mechanical properties of the tensioning polymer film to allow it especially to follow facial expressions without cracking, and thus to improve the remanence of the tensioning effect on the skin.

### DETAILED DESCRIPTION OF THE PREFERRED EMBODIMENTS

One subject of the present invention is a composition comprising, in a physiologically acceptable medium, a tensioning polymer and organic-mineral hybrid particles.

A subject of the invention is also a process for caring for the skin, more particularly facial skin, in particular wrinkled skin, comprising the topical applications to the skin of a composition as defined above.

The composition and the process according to the invention are in particular intended for smoothing out human facial and/or bodily skin and/or for reducing or effacing the signs of ageing of the skin, in particular for reducing or effacing skin wrinkles and/or fine lines.

Preferred constituents of the composition according to the invention will now be described in greater detail.

### Tensioning Polymer

According to the invention, the term “tensioning polymer” means a polymer that can have a tensioning effect, i.e. that can make the skin taut and, by means of this tensioning effect, smooth out the skin and reduce the wrinkles and fine lines or even make them disappear immediately.

More particularly, this expression denotes any polymer that is soluble or dispersible in water at a temperature ranging from 25° C. to 50° C. at a concentration of 7% by weight in water or at the maximum concentration at which a medium of uniform appearance is formed and producing at this concentration of 7% or at this maximum concentration in water a shrinkage of more than 15% in the test described below.

The maximum concentration at which a medium of uniform appearance forms is determined to within ±10% and preferably to within ±5%.

The expression “medium of uniform appearance” means a medium that does not contain any aggregates that are visible to the naked eye.

For the determination of the maximum concentration, the tensioning polymer is gradually added to the water with deflocculating stirring at a temperature ranging from 25° C. to 50° C., and the mixture is then stirred for one hour. The mixture thus prepared is then examined after 24 hours to see if it is of uniform appearance (absence of aggregates visible to the naked eye).

The tensioning effect may be characterized by an in vitro shrinkage test.

A homogeneous mixture of the tensioning polymer in water, at a concentration of 7% by weight or at the maximum concentration defined above, is prepared beforehand and as described previously.

30 μl of the homogeneous mixture are placed on a rectangular sample (10×40 mm, thus having an initial width L₀ of 10 mm) of elastomer with a modulus of 20 MPa and a thickness of 100 μm.

After drying for 3 hours at 22±3° C. and 40±10% relative humidity RH, the elastomer sample has a shrunken width, noted L₃ₕ, due to the tension exerted by the applied tensioning polymer.

The tensioning effect (TE) of the polymer is then quantified in the following manner:

$${}_{}^{‵}{TE}{}_{}^{ ′} = ( {L}_{0} - {L}_{3 h} / {L}_{0} ) \times 100 as \%$$

$$with {L}_{0} = initial width 10 mm$$

$$and {L}_{3 h} = width after 3 hours of drying$$

The tensioning polymer may be chosen especially from synthetic polymers, plant proteins and hydrolysates thereof, and mixtures thereof.

These various categories of tensioning polymers will now be described.

### a) Synthetic Polymers

The term “synthetic polymer” characterizes any polymer obtained chemically or via production in an organism of the components required for this production.

The synthetic tensioning polymer may be, in the composition according to the invention, either dissolved or dispersed in a polar or a polar liquid. It may in particular be dissolved in water or may be in the form of an aqueous particle dispersion.

The tensioning polymers may especially be in the form of interpenetrating polymer networks (IPNs), of polycondensates, acrylic polymers, grafted silicone polymers or star polymers.

### Interpenetrating Polymer Network

According to a first variant, the composition according to the present invention comprises at least one synthetic tensioning polymer of interpenetrating polymer network type.

For the purposes of the present invention, the expression “interpenetrating polymer network” means a blend of two interlaced polymers, obtained by simultaneous polymerization and/or crosslinking of two types of monomer, the blend obtained having a single glass transition temperature.

Examples of IPNs that are suitable for use in the present invention, and also the process for preparing them, are described in patents U.S. Pat. No. 6,139,322 and U.S. Pat. No. 6,465,001, for example.

Preferably, the IPN according to the invention comprises at least one polyacrylic polymer and more preferably also comprises at least one polyurethane or one copolymer of vinylidene fluoride and of hexafluoropropylene.

According to one preferred form, the IPN according to the invention comprises a polyurethane polymer and a polyacrylic polymer. Such IPNs are especially those of the Hybridur series that are commercially available from the company Air Products.

An IPN that is particularly preferred is in the form of an aqueous dispersion of particles with a weight-average size of between 90 and 110 nm and a number-average size of about 80 nm. This IPN preferably has a glass transition temperature, Tg, ranging from about −60° C. to +100° C. An IPN of this type is sold especially by the company Air Products under the trade name Hybridur X-01602. Another IPN that is suitable for use in the present invention is referenced Hybridur X18693-21.

Other IPNs that are suitable for use in the present invention comprise IPNs consisting of a blend of a polyurethane with a copolymer of vinylidene fluoride and of hexafluoropropylene. These IPNs may be prepared especially as described in patent U.S. Pat. No. 5,349,003. As a variant, they are commercially available in the form of a colloidal dispersion in water, in a ratio of the fluorinated copolymer to the acrylic polymer of between 70:30 and 75:25, under the trade names Kynar RC-10147 and Kynar RC-10151 from the company Atofina.

### Polycondensate

According to a second variant, the composition may comprise as synthetic tensioning polymer at least one polycondensate.

Polymers in the form of polycondensates with an anti-wrinkle effect have been described especially in patent application WO 98/29092.

Polycondensates that may be mentioned include polyurethanes, especially anionic, cationic, nonionic or amphoteric polyurethanes, polyurethane-acrylics, polyurethane-polyvinylpyrrolidones, polyester-poly-urethanes, polyether-polyurethanes and polyureas, and mixtures thereof.

The polyurethane may be, for example, an aliphatic, cycloaliphatic or aromatic polyurethane, or polyurea/urethane or polyurea copolymer, comprising, alone or as a mixture:
at least one block of linear or branched aliphatic and/or cycloaliphatic and/or aromatic polyester origin, and/or
at least one block of aliphatic and/or cyclo-aliphatic and/or aromatic polyether origin, and/or
at least one block comprising fluoro groups.

The polyurethanes may also be obtained from branched or unbranched polyesters, or from alkyds comprising labile hydrogens that are modified by reaction with a diisocyanate and a difunctional (for example dihydro, diamino or hydroxyamino) organic compound, also comprising either a carboxylic acid or carboxylate group, or a sulfonic acid or sulfonate group, or alternatively a neutralizable tertiary amine group or a quaternary ammonium group. Mention may also be made of polyesters, polyesteramides, fatty-chain polyesters, polyamides and epoxyester resins.

For the purpose of forming a polyurethane, as monomers bearing an anionic group that may be used in the polycondensation, mention may be made of dimethylol-propionic acid, trimellitic acid or a derivative such as trimellitic anhydride, the sodium salt of pentane-diol-3-sulfonic acid or the sodium salt of 5-sulfo-1,3-benzenedicarboxylic acid.

Among the polycondensates that may be mentioned are the polymers sold under the trade names Avalure UR405 and Avalure UR450 by the company Noveon.

### Acrylic Polymers

The tensioning polymer according to the invention may be chosen from linear statistical acrylic copolymers with a molecular weight of less than 600 000 g/mol and preferably with a weight-average molecular weight of between 15 000 and 600 000 g/mol and containing at least 70% of a monomer with a glass transition temperature Tg of greater than 40° C. (preferably >60° C.), the corresponding homopolymer of which is insoluble in water at 25° C. and at least one (meth)acrylic acid monomer. This copolymer may also contain a minor monomer with a Tg of less than 40° C.

These copolymers generally have an overall glass transition temperature of greater than or equal to 45° C.

The copolymers preferably used are those constituted of:
70% to 90% by weight of at least one monomer chosen from styrene, benzyl acrylate, cyclohexyl acrylate, tert-butyl acrylate, isobornyl acrylate, norbornyl acrylate, methyl methacrylate, ethyl methacrylate, isobutyl methacrylate, cyclohexyl methacrylate, benzyl methacrylate, tert-butyl methacrylate, isobornyl methacrylate and norbornyl methacrylate;
10% to 30% by weight of (meth)acrylic acid.

Preferentially, the copolymers used are constituted of:
70% to 90% by weight of at least one monomer chosen from styrene, methyl methacrylate and cyclohexyl methacrylate;
10% to 30% by weight of (meth)acrylic acid.

Among the polymers mentioned above, the ones that will be particularly preferred are:
methyl methacrylate/methacrylic acid copolymers; methyl methacrylate/acrylic acid copolymers, the copolymers containing between 70% and 90% by weight of methyl methacrylate;
ethyl methacrylate/methacrylic acid copolymers; ethyl methacrylate/acrylic acid copolymers, the copolymers containing between 70% and 90% by weight of ethyl methacrylate;
isobutyl methacrylate/methacrylic acid copolymers; isobutyl methacrylate/acrylic acid copolymers, the copolymers containing between 70% and 90% by weight of isobutyl methacrylate;
benzyl methacrylate/methacrylic acid copolymers; benzyl methacrylate/acrylic acid copolymers, the copolymers containing between 70% and 90% by weight of benzyl methacrylate;
benzyl acrylate/methacrylic acid copolymers; benzyl acrylate/acrylic acid copolymers, the copolymers containing between 70% and 90% by weight of benzyl acrylate;
cyclohexyl methacrylate/methacrylic acid copolymers; cyclohexyl methacrylate/acrylic acid copolymers, the copolymers containing between 70% and 90% by weight of cyclohexyl methacrylate;
cyclohexyl acrylate/methacrylic acid copolymers; cyclohexyl acrylate/acrylic acid copolymers, the copolymers containing between 70% and 90% by weight of cyclohexyl acrylate;
tert-butyl methacrylate/methacrylic acid copolymers; tert-butyl methacrylate/acrylic acid copolymers, the copolymers containing between 70% and 90% by weight of tert-butyl methacrylate;
tert-butyl acrylate/methacrylic acid copolymers; tert-butyl acrylate/acrylic acid copolymers, the copolymers containing between 70% and 90% by weight of tert-butyl acrylate;
isobornyl methacrylate/methacrylic acid copolymers; isobornyl methacrylate/acrylic acid copolymers, the copolymers containing between 70% and 90% by weight of isobornyl methacrylate;
isobornyl acrylate/methacrylic acid copolymers; isobornyl acrylate/acrylic acid copolymers, the copolymers containing between 70% and 90% by weight of isobornyl acrylate;
norbornyl methacrylate/methacrylic acid copolymers; norbornyl methacrylate/acrylic acid copolymers, the copolymers containing between 70% and 90% by weight of norbornyl methacrylate;
norbornyl acrylate/methacrylic acid copolymers; norbornyl acrylate/acrylic acid copolymers, the copolymers containing between 70% and 90% by weight of norbornyl acrylate; and
styrene/methacrylic acid copolymers; styrene/acrylic acid copolymers, the copolymers containing between 70% and 90% by weight of styrene.

The acrylic polymers sold under the names Neocryl XK 90 and Neocryl XK 99 by the company Avecia Neoresins may also be used.

### Grafted Silicone Polymer

Among the synthetic tensioning polymers used in the composition according to the invention, mention may be made, as a variant, of grafted silicone polymers, especially as defined in patent application EP-1 038 519. Such a polymer may be more particularly a polymer comprising a silicone or polysiloxane (Si—O-polymer) main chain onto which is grafted, within the chain and also optionally on at least one of its ends, at least one organic group not comprising silicone.

The polymers containing a polysiloxane backbone grafted with non-silicone organic monomers, according to the invention, can be existing commercial products or alternatively can be obtained according to any means known to those skilled in the art, in particular by reaction between (i) a starting silicone which is correctly functionalized on one or more of its silicon atoms, and (ii) a non-silicone organic compound which is itself correctly functionalized with a function which is capable of reacting with the functional group(s) borne by the silicone, forming a covalent bond; a classic example of such a reaction is the hydrosilylation reaction between =—Si—H groups and vinyl groups CH₂═CH—, or alternatively the reaction between thio functional groups —SH with these same vinyl groups.

Examples of polymers containing a polysiloxane backbone grafted with non-silicone organic monomers that are suitable for carrying out the present invention, and also their particular mode of preparation, are described in particular in patent applications EP-A-0 582 152, WO 93/23009 and WO 95/03776, the teachings of which are included in their entirety in the present description by way of non-limiting references.

According to a particularly preferred embodiment of the present invention, the silicone polymer containing a polysiloxane backbone grafted with non-silicone organic monomers which is used comprises the result of the free-radical copolymerization between, on the one hand, at least one non-silicone ethylenically unsaturated anionic organic monomer containing and/or a non-silicone ethylenically unsaturated hydrophobic organic monomer containing, and, on the other hand, a silicone containing in its chain at least one functional group capable of reacting with the ethylenic unsaturations of the non-silicone monomers, forming a covalent bond, in particular thio functional groups.

According to the present invention, the ethylenically unsaturated anionic monomers are preferably chosen, alone or as mixtures, from linear or branched, unsaturated carboxylic acids, optionally partially or totally neutralized in the form of a salt, it being possible for this or these unsaturated carboxylic acid(s) to be, more particularly, acrylic acid, methacrylic acid, maleic acid, maleic anhydride, itaconic acid, fumaric acid and crotonic acid. The suitable salts are, in particular, alkali metal salts, alkaline-earth metal salts and ammonium salts. It will likewise be noted that, in the final grafted silicone polymer, the organic group of anionic nature which comprises the result of the free-radical (homo)polymerization of at least one anionic monomer of unsaturated carboxylic acid type can, after reaction, be post-neutralized with a base (sodium hydroxide, aqueous ammonia, etc.) in order to place it in the form of a salt.

According to the present invention, the ethylenically unsaturated hydrophobic monomers containing are preferably chosen, alone or as mixtures, from acrylic acid esters of alkanols and/or methacrylic acid esters of alkanols. The alkanols are preferably C₁-C₁₈ and more particularly C₁-C₁₂. The preferred monomers are chosen from the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate and stearyl (meth)acrylate, or mixtures thereof.

One family of silicone polymers containing a polysiloxane backbone grafted with non-silicone organic monomers that is particularly suitable for carrying out the present invention consists of silicone polymers comprising in their structure the unit of formula (I) below:

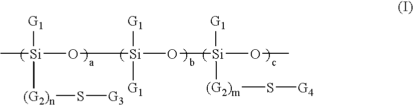

in which the radicals G₁, which may be identical or different, represent hydrogen, a C₁-C₁₀ alkyl radical or a phenyl radical; the radicals G₂, which may be identical or different, represent a C₁-C₁₀ alkylene group; G₃ represents a polymer residue resulting from the (homo)polymerization of at least one ethylenically unsaturated anionic monomer; G₄ represents a polymer residue resulting from the (homo)polymerization of at least one ethylenically unsaturated hydrophobic monomer; m and n are, independently of each other, equal to 0 or 1; a is an integer ranging from 0 to 50; b is an integer which may be between 10 and 350, c is an integer ranging from 0 to 50; with the proviso that one of the parameters a and c is other than 0.

Preferably, the unit of formula (I) above has at least one, and even more preferentially all, of the following characteristics:
the radicals G₁ denote a C₁-C₁₀ alkyl radical;
n is not zero, and the radicals G₂ represent a divalent C₁-C₃ radical;
G₃ represents a polymer radical resulting from the (homo)polymerization of at least one monomer of the ethylenically unsaturated carboxylic acid type, preferably acrylic acid and/or methacrylic acid;
G₄ represents a polymer radical resulting from the (homo)polymerization of at least one monomer of the C₁-C₁₀ alkyl (meth)acrylate type.

Examples of grafted silicone polymers corresponding to formula (I) are thus, especially, polydimethylsiloxanes (PDMSs) onto which are grafted, via a connecting chain thiopropylene type, mixed polymer units of the poly(meth)acrylic acid type and/or of the polyalkyl (meth)acrylate type.

These polymers are referenced under the CTFA name Polysilicone-8.

It may thus be a case of a polydimethylsiloxane grafted with propylthio(polymethyl acrylate/methyl methacrylate/methacrylic acid) or a polydimethyl-siloxane grafted with propylthio(polymethyl acrylate), propylthio(polymethyl methacrylate) and propylthio(polymethacrylic acid). As a variant, it may be a polydimethylsiloxane grafted with propylthio(polyisobutyl methacrylate) and propylthio(polymethacrylic acid). A polydimethyl-siloxane grafted with propylthio(polymethyl acrylate/methyl methacrylate/methacrylic acid) is preferably used.

Such grafted silicone polymers are especially sold by the company 3M under the trade names VS 80, VS 70 and LO21.

Preferably, the number-average molecular mass of the silicone polymers containing a polysiloxane backbone grafted with non-silicone organic monomers of the invention ranges from 10 000 to 1 000 000 approximately and even more preferentially from 10 000 to 100 000 approximately.

### Star Polymer

According to yet another possibility, the synthetic tensioning polymer that may be used in the composition according to the invention may comprise at least one polymer of “star” structure represented by formula (II) below:

A-[(M₁)ₚ₁-(M₂)ₚ₂ . . . (Mᵢ)ₚⱼ]ₙ (II)

in which:
A represents a multifunctional centre, of functionality “n”, n being an integer greater than 2, in particular greater than 5,
[(M₁)ₚ₁-(M₂)ₚ₂ . . . (Mᵢ)ₚⱼ] represents a polymer chain, also referred to as a “branch”, consisting of polymerized monomers Mᵢ, which may be identical or different, having a polymerization index pj, each branch being identical or different, and being covalently grafted onto the centre A,
i is greater than or equal to 1, and pj is greater than or equal to 2; the polymer comprising one or more monomers Mᵢ whose corresponding homopolymer has a Tg of greater than or equal to about 10° C., preferably greater than or equal to 15° C. and better still greater than or equal to 20° C.; and this or these monomer(s) Mᵢ being present in a minimum amount of about 45% by weight, preferably in an amount of between 55% and 99% by weight and better still in an amount of between 75% and 90% by weight, relative to the total weight of monomers of the final polymer. These polymers, and the process for preparing them, are described in particular in the document EP-1 043 345.

### b) Plant Proteins and Hydrolysates Thereof.

Examples of plant proteins and plant protein hydrolysates that may be used as tensioning agents according to the invention are constituted of proteins and protein hydrolysates of corn, rye, wheat, buckwheat, sesame, spelt, pea, bean, lentil, soybean and lupin.

Advantageously, the tensioning polymer is chosen from synthetic polymers of interpenetrating polymer network type, especially interpenetrating networks of polyurethane and of polyacrylic, and silicone polymers containing a polysiloxane backbone grafted with non-silicone organic monomers, as described previously.

The tensioning polymer may be included in the composition according to the invention in a content ranging from 0.01% to 20% by weight of active material, preferably from 1% to 10% by weight of active material, relative to the total weight of the composition. The term “active material” means the polymer without the medium in which it is possibly dissolved or dispersed.

### Organic-Mineral Hybrid Particles

The expression “organic-mineral hybrid particles” means composite particles constituted of at least one synthetic polymer constituting a matrix for these particles, and of at least two colloidal mineral particles included in the matrix of these particles.

The term “colloidal mineral particles” means particles of mineral fillers with a number-average diameter ranging from 0.1 to 100 nm and preferably from 3 to 30 nm.

The colloidal mineral particles may be chosen from colloidal particles of silica, cerium oxide, zirconium oxide, alumina, calcium carbonate, barium sulfate, calcium sulfate, zinc oxide and titanium dioxide, colloidal platinum particles, mixed colloidal particles, for instance silica-coated titanium dioxides, or silica-alumina composite colloidal particles. Colloidal silica particles or silica-alumina composite colloidal particles will preferably be used.

In one alternative embodiment of the invention, the colloidal mineral particles may be chosen from mixed silicate colloidal particles. Phyllosilicates will then preferably be used, i.e. silicates having a structure in which the SiO₄ tetrahedra are organized in sheets between which are enclosed the metal cations. One family of silicates that is particularly preferred is the family of laponites. Laponites are magnesium lithium sodium silicates having a layered structure similar to that of montmorillonites. Laponite is the synthetic form of the natural mineral known as hectorite.

The colloidal mineral particles preferably constitute from 5% to 80% by weight, preferably from 20% to 80% by weight and preferentially from 30% to 70% by weight relative to the total weight of the organic-mineral hybrid particles.

These colloidal mineral particles may be present in the hybrid particles according to the invention in the form of total, partial or surface inclusions.

The polymers constituting the matrix of the organic-mineral hybrid particles may be chosen for example from polyurethanes; free-radical polymers including acrylic polymers, and preferably from acrylic polymers.

The free-radical polymers may especially comprise one or more monomers chosen from (meth)acrylic acids, ethylene, styrene, methylstyrene, esters of vinyl alcohol and of a C₁-C₁₈ monocarboxylic acid, for instance vinyl acetate, vinyl propionate, vinyl n-butyrate, vinyl laurate or vinyl stearate; maleic acid, fumaric acid, itaconic acid, C₁-C₈ (preferably C₁-C₄) alkyl (meth)acrylates, for instance methyl (meth)acrylate, ethyl (meth)acrylate, n-butyl (meth)acrylate, isobutyl (meth)acrylate or 2-ethylhexyl (meth)acrylate. Preferably, the free-radical polymers comprise one or more monomers chosen from C₁-C₈ (especially C₁-C₄) alkyl (meth)acrylates.

The organic-mineral hybrid particles may comprise from 20% to 95% by weight, preferably from 20% to 80% by weight and preferentially from 30% to 70% by weight, relative to the weight of the particles, of synthetic polymer (forming the matrix of the particles). The remainder to 100% by weight is constituted of colloidal mineral particles.

Preferably, the organic-mineral hybrid particles are chosen from acrylic polymer-silica hybrid particles.

Advantageously, the organic-mineral hybrid particles have a volume-average diameter ranging from 0.03 to 1 μm and preferentially from 0.05 to 0.5 μm. They may be in the form of a solution or as a suspension in a polar or apolar liquid, or alternatively in dry form redispersible in a cosmetic solvent.

Advantageously, the organic-mineral hybrid particles are in the form of an aqueous dispersion.

The organic-mineral hybrid particles may be present in the composition in an active material amount ranging from 0.01% to 30% and preferably from 1% to 20% of the total weight of the composition.

Such organic-mineral hybrid particles are especially described in patent applications WO 2006/048 167, WO 2006/072 464 and US 2004/0 171 728. They are especially formed by emulsion polymerization of the monomers of the polymer forming the matrix in the presence of colloidal mineral particles.

Examples of organic-mineral hybrid particles that may be mentioned include silica-acrylic copolymer composite particles in aqueous dispersion, such as those sold under the names Acnano DA 1000× and Nanocomposite R2/768 by the company BASF.

The composition according to the invention is preferably generally suited to topical application to the skin and thus generally comprises a physiologically acceptable medium, i.e. a medium that is compatible with the skin and/or its integuments. It is preferably a cosmetically acceptable medium, i.e. a medium that has a pleasant colour, odour and feel and that does not cause any unacceptable discomfort (stinging, tautness or redness) liable to put the consumer off using this composition.

The composition according to the invention may be in any galenical form, including those conventionally used for topical application, and especially in the form of dispersions of the lotion or aqueous gel type, emulsions of liquid or semi-liquid consistency of the milk type, obtained by dispersing a fatty phase in an aqueous phase (O/W) or, conversely, (W/O), or suspensions or emulsions of soft, semi-solid or solid consistency of the cream or gel type, or alternatively multiple emulsions (W/O/W or O/W/O), microemulsions, vesicular dispersions of ionic and/or nonionic type, or wax/aqueous phase dispersions. These compositions are prepared according to the usual methods.

According to one preferred embodiment of the invention, the composition is in the form of an O/W emulsion or an aqueous gel.

This composition may also contain various adjuvants commonly used in cosmetics, such as emulsifiers, for instance fatty acid esters of polyethylene glycol, optionally polyoxyethylenated fatty acid esters of sorbitan, polyoxyethylenated fatty alcohols, and fatty acid esters or ethers of sugars such as sucrose or glucose; fillers; preserving agents; sequestrants; fragrances; and thickeners and/or gelling agents, in particular polyacrylamides, acrylic homopolymers and copolymers, and acrylamidomethylpropanesulfonic acid homopolymers and copolymers.

Needless to say, a person skilled in the art will take care to select this or these optional additional compound(s) and/or the amount thereof such that the anti-wrinkle properties of the composition according to the invention are not, or are not substantially, adversely affected by the envisaged addition.

The composition according to the invention may also contain anti-ageing active agents with an effect complementary to the combination according to the invention, such as at least one compound chosen from desquamating agents, moisturizers, agents for stimulating keratinocyte proliferation and/or differentiation, agents for stimulating collagen and/or elastin synthesis or for preventing their degradation, depigmenting agents, anti-glycation agents, agents for stimulating glycosaminoglycan synthesis, dermo-decontracting agents or muscle relaxants, antioxidants and free-radical scavengers, and mixtures thereof.

Examples of such active agents are: retinol and derivatives thereof such as retinyl palmitate; ascorbic acid and derivatives thereof such as magnesium ascorbyl phosphate and ascorbyl glucoside; tocopherol and derivatives thereof such as tocopheryl acetate; nicotinic acid and precursors thereof such as nicotinamide; ubiquinone; glutathione and precursors thereof such as L-2-oxothiazolidine-4-carboxylic acid; plant extracts and especially extracts of sea fennel and of olive leaf; algal extracts and in particular of laminaria; bacterial extracts; sapogenins such as diosgenin and extracts of Dioscorea plants, in particular of wild yam, containing them; α-hydroxy acids; β-hydroxy acids, such as salicylic acid and 5-n-octanoylsalicylic acid; oligopeptides and pseudodipeptides and acyl derivatives thereof, in particular {2-[acetyl(3-trifluoromethylphenyl)amino]-3-methyl-butyrylamino}acetic acid and the lipopeptides sold by the company Sederma under the trade names Matrixyl 500 and Matrixyl 3000; lycopene; and mixtures thereof.

The composition according to the invention is applied according to the usual techniques, for example by application (especially of creams, gels, sera or lotions) to the skin intended to be treated, in particular the skin of the face and/or the neck, especially the skin around the eyes. In the context of this process, the composition may be, for example, a care composition or a makeup composition, in particular a foundation.

The invention will now be described with reference to the following examples, which are given as non-limiting illustrations.

### EXAMPLES

### Examples 1 and 2

### Cosmetic Compositions

### Anti-Wrinkle Sera

Two sera according to the invention (Examples 1A and 2A) and two sera not forming part of the invention (Examples 1B and 2B), the latter sera not containing organic-mineral hybrid particles, were prepared.

### Example 1A

### Invention

Anti-wrinkle serum containing 3.5% Hybridur and 3.5% of organic-mineral hybrid particles.

Phase A

| | | |
|---|---|---|
| Water | 76.90 | g |
| Methyl vinyl ether/maleic anhydride | 0.20 | g |
| copolymer crosslinked with 1,9-decadiene | | |
| (Antaron ST 06 from ISP) | | |
| Xanthan gum (Keltrol T from Kelco) | 0.20 | g |
| Preserving agent | 0.55 | g |

Phase B

| | | |
|---|---|---|
| Water | 1.00 | g |
| Triethanolamine | 0.20 | g |

Phase C

| | | |
|---|---|---|
| Acrylamide/sodium acrylamido-2-methyl- | 1.00 | g |
| propanesulfonate copolymer as an inverse | | |
| emulsion at 40% in isoparaffin/water | | |
| (Sepigel 305 from SEPPIC) | | |
| Preserving agent | 0.30 | g |

Phase D

| | | |
|---|---|---|
| Aqueous dispersion containing 40% by weight | 8.63 | g |
| of particles of an interpenetrating polymer | | |
| network of polyurethane and polyacrylic | | |
| polymers (Hybridur 875 from Air Products) | | |
| Aqueous dispersion of acrylic polymer-silica | 10.00 | g |
| hybrid particles (AcNano DS 1000 X from BASF) | | |

### Example 1B

### Outside the Invention

Anti-wrinkle serum containing 7% Hybridur 875.

Phase A

| | | |
|---|---|---|
| Water | 78.25 | g |
| Methyl vinyl ether/maleic anhydride | 0.20 | g |
| copolymer crosslinked with 1,9-decadiene | | |
| (Antaron ® ST 06 from ISP) | | |
| Xanthan gum (Keltrol ® T from Kelco) | 0.20 | g |
| Preserving agent | 0.55 | g |

Phase B

| | | |
|---|---|---|
| Water | 1.00 | g |
| Triethanolamine | 0.20 | g |

Phase C

| | | |
|---|---|---|
| Acrylamide/sodium acrylamido-2-methyl - | 1.00 | g |
| propanesulfonate copolymer as an inverse | | |
| emulsion at 40% in isoparaffin/water | | |
| (Sepigel ® 305 from SEPPIC) | | |
| Preserving agent | 0.30 | g |

Phase D

| | | |
|---|---|---|
| Aqueous dispersion containing 40% by weight | 7.30 | g |
| of particles of an interpenetrating polymer | | |
| network of polyurethane and polyacrylic | | |
| polymers (Hybridur ® 875 from Air Products) | | |

The anti-wrinkle serum was prepared in the following manner:

Phase A is heated to about 75° C., with stirring, and phase B is then poured into phase A. Next, the heating is stopped, while continuing the stirring until the mixture has cooled to room temperature, and phases C and D are then added. Gentle stirring is then maintained for 30 minutes.

### Example 2A

### Invention

Anti-wrinkle serum containing 3.5% Avalure and 3.5% of organic-mineral hybrid particles.

Phase A

| | | |
|---|---|---|
| Water | 75.55 | g |
| Methyl vinyl ether/maleic anhydride | | |
| copolymer crosslinked with 1,9-decadiene | 0.20 | g |
| (Antaron ® ST 06 from ISP) | | |
| Xanthan gum (Keltrol ® T from Kelco) | 0.20 | g |
| Preserving agent | 0.55 | g |

Phase B

| | | |
|---|---|---|
| Water | 1.00 | g |
| Triethanolamine | 0.20 | g |

Phase C

| | | |
|---|---|---|
| Acrylamide/sodium acrylamido-2-methyl- | 1.00 | g |
| propanesulfonate copolymer as an inverse | | |
| emulsion at 40% in isoparaffin/water | | |
| (Sepigel ® 305 from SEPPIC) | | |
| Preserving agent | 0.30 | g |

Phase D

| | | |
|---|---|---|
| Aqueous dispersion containing 35% polyurethane | 10.00 | g |
| (Avalure ® UR405 from Noveon) | | |
| Aqueous dispersion of acrylic polymer-silica | 10.00 | g |
| hybrid particles (AcNano DS 1000 X from BASF) | | |

### Example 2B

### Outside the Invention

Anti-wrinkle serum containing 3.5% Avalure UR405.

Phase A

| | | |
|---|---|---|
| Water | 85.55 | g |
| Methyl vinyl ether/maleic anhydride | 0.20 | g |
| copolymer crosslinked with 1,9-decadiene | | |
| (Antaron ® ST 06 from ISP) | | |
| Xanthan gum (Keltrol ® T from Kelco) | 0.20 | g |
| Preserving agent | 0.55 | g |

Phase B

| | | |
|---|---|---|
| Water | 1.00 | g |
| Triethanolamine | 0.20 | g |

Phase C

| | | |
|---|---|---|
| Acrylamide/sodium acrylamido-2-methyl- | 1.00 | g |
| propanesulfonate copolymer as an inverse | | |
| emulsion at 40% in isoparaffin/water | | |
| (Sepigel ® 305 from SEPPIC) | | |
| Preserving agent | 0.30 | g |

Phase D

| | | |
|---|---|---|
| Aqueous dispersion containing 35% polyurethane | 10.00 | g |
| (Avalure ® UR405 from Noveon) | | |

### Example 3

### Demonstration of the Improvement of the Remanence Properties of the Formulations According to the Invention

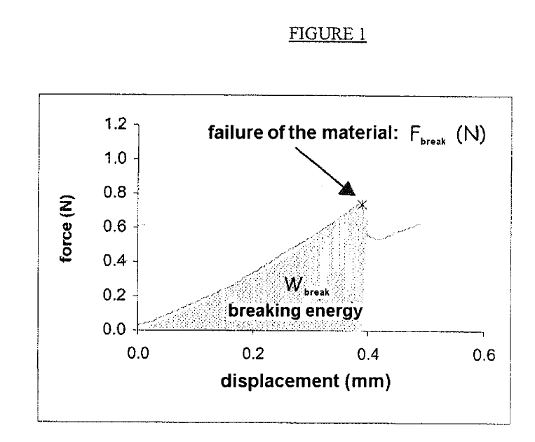

The test consists in subjecting to compression to the point of failure a material (in this instance the anti-wrinkle creams of Examples 1A and 1B) placed on the surface of a soft deformable foam. The use of this foam support makes it possible to impose a large deformation on the material placed on the surface, and thus to quantify its breaking strength. The mechanical compression stress is exerted using a cylindrical punch 1 mm in diameter; the speed of travel of the punch being 0.1 mm/s. The test is performed using a TA-XT21 texture analyser sold by the company Stable Micro System. A curve of the force F (in N) as a function of the displacement d (in mm) is thus obtained, from which it is possible to determine the point of failure of the material. The attached FIG. 1 shows such an example of a curve of force as a function of displacement.

The parameter Wbreak (the breaking energy in J/m²) is used to quantity the breaking strength of the material. The parameter corresponding to the area under the curve F=f(d)/area of the punch.

The substrate consists of a neoprene foam 13 mm thick. The material (anti-wrinkle composition) is placed on this substrate so as to obtain, after drying for 24 hours, a film 15 to 30 μm thick. The deposits were produced using a film spreader that deposits 650 μm wet.

The results obtained are as follows:

| | W_{break} |
|---|---|
| Material | (J/m²) |
| Example 1A | 417 ± 38 |
| Serum containing 3.5% Hybridur 875 and | |
| 3.5% of organic-mineral hybrid particles | |
| (AcNano DS 1000 X from BASF | |
| Example 1B (comparative) | 59 ± 7 |
| Serum containing 7% Hybridur 875 | |
| Example 2A | 1978 ± 520 |
| Serum containing 3.5% Avalure UR405 and | |
| 3.5% of organic-mineral hybrid particles | |
| (AcNano DS 1000 X from BASF) | |
| Example 2B (comparative) | 395 ± 30 |
| Serum containing 3.5% Avalure UR405 | |

It is found that the hybrid particles used in the compositions according to the invention of Examples 1A and 2A have a reinforcing role, this reinforcing role being illustrated by an increase in the breaking energy. This results in a more remanent tensioning effect of the compositions of Examples 1A and 2A in comparison with the tensioning effect of compositions 1B and 2B not forming part of the invention. Specifically, if the tensioning effect is associated with the formation of a rigid deposit, it is understood that the perennity of this effect is associated with the mechanical strength of this deposit.

It was moreover confirmed on a panel of 6 women that the compositions of Examples 1A and 2A have a satisfactory tensioning effect.

### Example 4

Anti-wrinkle serum containing 3.5% grafted silicone polymer and 3.5% of organic-mineral hybrid particles.

The following composition is prepared:

Phase A

| Water | qs 100 | g |
|---|---|---|
| Methyl vinyl ether/maleic anhydride copolymer | 0.20 | g |
| crosslinked with 1,9-decadiene | | |
| (Antaron ST 06 from ISP) | | |
| Xanthan gum (Keltrol T from Kelco) | 0.20 | g |
| Preserving agent | 0.55 | g |

Phase B

| | | |
|---|---|---|
| Water | 1.00 | g |
| Triethanolamine | 0.20 | g |

Phase C

| | | |
|---|---|---|
| Acrylamide/sodium acrylamido-2-methylpropane- | 1.00 | g |
| sulfonate copolymer as an inverse emulsion at | | |
| 40% in isoparaffin/water (Sepigel 305 from | | |
| SEPPIC) | | |
| Preserving agent | 0.30 | g |

Phase D

| | | |
|---|---|---|
| Polydimethyl/methyl siloxane containing methyl | 8.63 | g |
| 3-thiopropylacrylate/methyl methacrylate/ | | |
| methacrylic acid groups (LO21 Dry from 3M) | | |
| Aqueous dispersion of acrylic polymer- | 10.00 | g |
| silica hybrid particles (AcNano DS 1000 X | | |
| from BASF) | | |

The composition applied to the skin gives a long-lasting anti-wrinkle effect.

The invention method and composition is preferably used by subjects desirous of the benefits noted herein, subjects “in need of” these benefits.

The above written description of the invention provides a manner and process of making and using it such that any person skilled in this art is enabled to make and use the same, this enablement being provided in particular for the subject matter of the appended claims, which make up a part of the original description and including a composition comprising, in a physiologically acceptable medium, a tensioning polymer and organic-mineral hybrid particles.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising, in a physiologically acceptable medium, a tensioning polymer and organic-mineral hybrid particles.

2. The composition according to claim 1, wherein the tensioning polymer is chosen from synthetic polymers, plant proteins and hydrolysates thereof, and mixtures thereof.

3. The composition according to claim 1, wherein the tensioning polymer is included in the composition in a content ranging from 1 to 10 by weight of active material relative to the total weight of the composition.

4. The composition according to claim 1, wherein the tensioning polymer is chosen from silicone polymers comprising a polysiloxane backbone grafted with non-silicone organic monomers.

5. The composition according to claim 4, wherein the silicone polymer comprises in its structure the unit of formula (I) below: 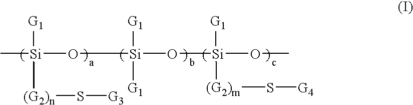
in which the radicals G₁, which may be identical or different, represent hydrogen, a C₁-C₁₀ alkyl radical or a phenyl radical; the radicals G₂, which may be identical or different, represent a C₁-C₁₀ alkylene group; G₃ represents a polymer residue resulting from the (homo)polymerization of at least one ethylenically unsaturated anionic monomer; G₄ represents a polymer residue resulting from the (homo)polymerization of at least one ethylenically unsaturated hydrophobic monomer; m and n are, independently of each other, equal to 0 or 1; a is an integer ranging from 0 to 50; b is an integer between 10 and 350, c is an integer ranging from 0 to 50; with the proviso that one of the parameters a and c is other than 0.

6. The composition according to claim 5, wherein the unit of formula (I) has at least one of the following characteristics:
the radicals G₁ denote a C₁-C₁₀ alkyl radical;
n is not zero, and the radicals G₂ represent a divalent C₁-C₃ radical;
G₃ represents a polymer radical resulting from the (homo)polymerization of at least one monomer of the ethylenically unsaturated carboxylic acid type, preferably acrylic acid and/or methacrylic acid;
G₄ represents a polymer radical resulting from the (homo)polymerization of at least one monomer of the C₁-C₁₀ alkyl (meth)acrylate type.

7. The composition according to claim 5, wherein the grafted silicone polymers corresponding to formula (I) are polydimethylsiloxanes (PDMSs) onto which are grafted, via a connecting chain of thiopropylene type, polymer units of the poly(meth)acrylic acid type and/or of the polyalkyl (meth)acrylate type, especially of C₁-C₃ or even C₁ alkyl.

8. The composition according to claim 4, wherein the grafted silicone polymer is a polydimethylsiloxane grafted with propylthio(polymethyl acrylate/methyl methacrylate/methacrylic acid).

9. The composition according to claim 1, wherein the tensioning polymer is an interpenetrating polymer network type polymer.

10. The composition according to claim 9, wherein the interpenetrating polymer network comprises a polyurethane polymer and an acrylic polymer.

11. The composition according to claim 1, wherein the organic-mineral hybrid particles comprise a synthetic polymer matrix in which are included at least colloidal mineral particles.

12. The composition according to claim 11, wherein the colloidal mineral particles have a number-average diameter ranging from 0.1 to 100 nm.

13. The composition according to claim 11, wherein the colloidal mineral particles are chosen from colloidal particles of silica, cerium oxide, zirconium oxide, alumina, calcium carbonate, barium sulfate, calcium sulfate, zinc oxide and titanium dioxide, colloidal platinum particles, mixed colloidal particles, for instance silica-coated titanium dioxides, or silica-alumina composite colloidal particles or mixed silicate colloidal particles.

14. The composition according to claim 11, wherein the colloidal mineral particles are chosen from colloidal silica particles and silica-alumina composite colloidal particles.

15. The composition according to claim 11, wherein the colloidal mineral particles constitute from 5% to 80% by weight relative to the total weight of the organic-mineral hybrid particles.

16. The composition according to claim 11, wherein the polymer constituting the matrix of the organic-mineral hybrid particles is chosen from polyurethanes and acrylic polymers.

17. The composition according to claim 11, wherein the polymer constituting the matrix of the organic-mineral hybrid particles comprises one or more polymerized monomers chosen from C₁-C₈ and preferably C₁-C₄ alkyl (meth)acrylates.

18. The composition according to claim 1, wherein the organic-mineral hybrid particles are chosen from acrylic polymer-silica hybrid particles.

19. The composition according to claim 1, wherein the organic-mineral hybrid particles have a volume-average diameter ranging from 0.03 to 1 μm.

20. The composition according to claim 1, wherein the organic-mineral hybrid particles are in the form of an aqueous dispersion.

21. The composition according to claim 1, wherein the organic-mineral hybrid particles are present in an active material amount ranging from 0.01% to 30% by weight relative to the total weight of the composition.

22. The composition according to claim 1, wherein said composition in the form of an O/W emulsion or an aqueous gel.

23. A skincare process, comprising the application to the skin of a composition according to claim 1.

24. The process according to claim 23, wherein the composition is applied to wrinkled skin.
